(19) United States
(12) Patent Application Publication
(10) Pub. No.: US 2026/0041317 A1
(43) Pub. Date: Feb. 12, 2026

(54) LARYNGOSCOPE HANDLE

(71) Applicant: BON SECOURS MERCY HEALTH, INC., Marriottsville, MD (US)

(72) Inventors: David Ledrick, Toledo, OH (US); Damion Vitt, Fostoria, OH (US)

(21) Appl. No.: 19/250,156

(22) Filed: Jun. 26, 2025

Related U.S. Application Data
(60) Provisional application No. 63/680,326, filed on Aug. 7, 2024.

(51) Int. Cl. A61B 1/267; A61B 1/00; A61B 1/06; A61B 90/00

(52) U.S. Cl. CPC A61B 1/267; A61B 1/00032; A61B 1/00066; A61B 1/00126; A61B 1/00128; A61B 1/00137; A61B 1/00167; A61B 1/0669; A61B 90/08; A61B 2090/0808

(57) ABSTRACT

A laryngoscope handle for use with a laryngoscope blade that is designed to promote an upward lifting motion and discourage titling or levering of the laryngoscope handle during direct or indirect laryngoscopy and endotracheal intubation is provided. The laryngoscope handle can include a cap disposed at a first end, a blade interface disposed at a second end, opposite the cap, and a gripping portion disposed between the cap and the blade interface. The cap can extend outward from the gripping portion at the first end. The cap can be angled differently than the blade interface relative to the longitudinal axis of the laryngoscope handle thereby encouraging a user to lift upward on the laryngoscope handle during performance of endotracheal intubation.

### CROSS-REFERENCE TO RELATED APPLICATIONS

This application claims the benefit of U.S. Provisional Application No. 63/680,326, filed on Aug. 7, 2024. The entire disclosure of the above application is incorporated herein by reference.

### FIELD

The present technology relates to medical devices for airway management, and, more particularly, to a laryngoscope handle configured to improve the operation, performance, and functionality of a laryngoscope used when performing endotracheal intubation.

### Introduction

This section provides background information related to the present disclosure which is not necessarily prior art.

A laryngoscope is a medical device used by a healthcare professional in emergency medicine to perform tracheal intubation, including direct or indirect laryngoscopy and endotracheal intubation, procedures to visualize the larynx and surrounding structures in the throat of a patient, and for ensuring patient airway patency. The laryngoscope can include a handle and a detachable blade for accessing and visualizing a patient's airway. Despite the widespread use of laryngoscopes, the design of laryngoscopes has not substantially evolved to enhance operability, which can hinder ease of use for the healthcare professional and can result in undesirable outcomes for the patient.

During direct or indirect laryngoscopy and endotracheal intubation, the medical professional can begin by positioning the patient appropriately—usually in a supine position with the patient's head slightly extended and supported to align the oral, pharyngeal, and tracheal axes. The medical professional can then insert the laryngoscope blade into the patient's mouth, advancing it along the surface of the tongue. Once the tip of the laryngoscope blade reaches the base of the tongue and the epiglottis is visualized, the laryngoscope can then be lifted upward and away from the patient at an angle of approximately 35 degrees to expose the vocal cords. However, due to the design of certain laryngoscope handles, the medical professional may inadvertently tilt or lever the laryngoscope blade against the patient's teeth or gums, which can result in undesirable outcomes such as chipped or broken teeth and internal soft tissue injury.

Accordingly, there is a need for a laryngoscope handle that is configured to promote an upward lifting motion and discourage titling or levering of the laryngoscope during direct or indirect laryngoscopy and endotracheal intubation.

### SUMMARY

In concordance with the instant disclosure, a laryngoscope handle that is configured to promote an upward lifting motion and discourage titling or levering of the laryngoscope during direct or indirect laryngoscopy and endotracheal intubation, has surprisingly been discovered. The present technology includes articles of manufacture, systems, and processes that relate to a laryngoscope handle that is configured to promote an upward lifting motion and discourage titling or levering of the laryngoscope during direct or indirect laryngoscopy and endotracheal intubation.

In certain embodiments, a laryngoscope handle for use with a laryngoscope blade is provided that can include a cap disposed at a first end, a blade interface disposed at a second end, opposite the cap, and a gripping portion disposed between the cap and the blade interface. The cap can extend outward from the gripping portion at the first end. The cap can be angled differently than the blade interface relative to the longitudinal axis of the laryngoscope handle thereby encouraging a user to lift or pull upward on the laryngoscope handle during performance of endotracheal intubation.

In certain embodiments, a laryngoscope system for use during tracheal intubation is provided. The laryngoscope system can include a laryngoscope handle as described herein. The laryngoscope system can also include an illumination system and a laryngoscope blade, where the illumination system can include a power supply and a light source.

In certain embodiments, a method of using a laryngoscope system for tracheal intubation is provided. The method can include a step of providing the laryngoscope system, which can include a laryngoscope handle and a laryngoscope blade as described herein. The method can include a step of coupling the laryngoscope blade to the blade interface of the laryngoscope handle. The method can include a step of adjusting the laryngoscope blade from a first position to a second position. The method can also include a step of illuminating a light source associated with the laryngoscope system. The method can include a step of inserting the laryngoscope blade into a mouth of a patient and advancing it into a throat area to reach an operative position. Once the laryngoscope blade is in the operative position, the method can include a step of lifting the laryngoscope handle in an upward and outward direction relative to the patient's body.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is a top perspective view of a laryngoscope handle, according to an embodiment of the present disclosure;

FIG. 2 is a bottom perspective view of the laryngoscope handle, according to the embodiment shown in FIG. 1;

FIG. 3 is a front elevational view of the laryngoscope handle, according to the embodiment shown in FIG. 1;

FIG. 4 is a rear elevation view of the laryngoscope handle, according to the embodiment shown in FIG. 1;

FIG. 5 is a top plan view of the laryngoscope handle, according to the embodiment shown in FIG. 1;

FIG. 6 is a top plan view of the of the laryngoscope handle with a battery cover removed, according to the embodiment shown in FIG. 1;

FIG. 7 is a bottom plan view of the laryngoscope handle, according to the embodiment shown in FIG. 1;

FIG. 8 is a right side view of the laryngoscope handle, according to the embodiment shown in FIG. 1;

FIG. 9 is a left side view of the laryngoscope handle, according to the embodiment shown in FIG. 1;

FIG. 10 is an exploded top perspective view of the laryngoscope handle, according to the embodiment shown in FIG. 1;

FIG. 11 is a cross-sectional view of the laryngoscope handle, according to the embodiment shown in FIG. 1;

FIG. 12 is a top perspective view of a laryngoscope system, according to another embodiment of the present disclosure;

FIG. 13 is a diagrammatic side elevational view of the laryngoscope system, according to the embodiment shown in FIG. 11, with the laryngoscope system being used to perform direct laryngoscopy; and

FIGS. 14 and 15 provide a flowchart illustrating a method of using a laryngoscope system for tracheal intubation, according to an embodiment of the present disclosure.

### DETAILED DESCRIPTION

The following description of technology is merely exemplary in nature of the subject matter, manufacture and use of one or more inventions, and is not intended to limit the scope, application, or uses of any specific invention claimed in this application or in such other applications as may be filed claiming priority to this application, or patents issuing therefrom. Regarding methods disclosed, the order of the steps presented is exemplary in nature, and thus, the order of the steps can be different in various embodiments, including where certain steps can be simultaneously performed, unless expressly stated otherwise. “A” and “an” as used herein indicate “at least one” of the item is present; a plurality of such items may be present, when possible. Except where otherwise expressly indicated, all numerical quantities in this description are to be understood as modified by the word “about” and all geometric and spatial descriptors are to be understood as modified by the word “substantially” in describing the broadest scope of the technology. “About” when applied to numerical values indicates that the calculation or the measurement allows some slight imprecision in the value (with some approach to exactness in the value; approximately or reasonably close to the value; nearly). If, for some reason, the imprecision provided by “about” and/or “substantially” is not otherwise understood in the art with this ordinary meaning, then “about” and/or “substantially” as used herein indicates at least variations that may arise from ordinary methods of measuring or using such parameters.

Although the open-ended term “comprising,” as a synonym of non-restrictive terms such as including, containing, or having, is used herein to describe and claim embodiments of the present technology, embodiments may alternatively be described using more limiting terms such as “consisting of” or “consisting essentially of.” Thus, for any given embodiment reciting materials, components, or process steps, the present technology also specifically includes embodiments consisting of, or consisting essentially of, such materials, components, or process steps excluding additional materials, components or processes (for consisting of) and excluding additional materials, components or processes affecting the significant properties of the embodiment (for consisting essentially of), even though such additional materials, components or processes are not explicitly recited in this application. For example, recitation of a composition or process reciting elements A, B and C specifically envisions embodiments consisting of, and consisting essentially of, A, B and C, excluding an element D that may be recited in the art, even though element D is not explicitly described as being excluded herein.

As referred to herein, disclosures of ranges are, unless specified otherwise, inclusive of endpoints and include all distinct values and further divided ranges within the entire range. Thus, for example, a range of “from A to B” or “from about A to about B” is inclusive of A and of B. Disclosure of values and ranges of values for specific parameters (such as amounts, weight percentages, etc.) are not exclusive of other values and ranges of values useful herein. It is envisioned that two or more specific exemplified values for a given parameter may define endpoints for a range of values that may be claimed for the parameter. For example, if Parameter X is exemplified herein to have value A and also exemplified to have value Z, it is envisioned that Parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if Parameter X is exemplified herein to have values in the range of 1-10, or 2-9, or 3-8, it is also envisioned that Parameter X may have other ranges of values including 1-9, 1-8, 1-3, 1-2, 2-10, 2-8, 2-3, 3-10, 3-9, and so on.

When an element or layer is referred to as being “on,” “engaged to,” “connected to,” or “coupled to” another element or layer, it may be directly on, engaged, connected or coupled to the other element or layer, or intervening elements or layers may be present. In contrast, when an element is referred to as being “directly on,” “directly engaged to,” “directly connected to” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

In accordance with the present disclosure, a laryngoscope handle 100, a laryngoscope system 200, and a method 300 of using a laryngoscope handle 100 are provided. Advantageously, the present disclosure addresses shortcomings in laryngoscopes by providing a laryngoscope handle 100 that is configured to promote an upward and outward lifting motion and discourage titling or levering of the laryngoscope handle 100 during direct or indirect laryngoscopy and endotracheal intubation. The laryngoscope handle 100 can allow a medical professional to more accurately and easily perform direct or indirect laryngoscopy and endotracheal intubation, while helping to avoid undesirable patient outcomes such as chipped or broken teeth and internal soft tissue injury. The laryngoscope handle 100 can also militate against hand and wrist fatigue, enabling the medical professional to perform intubations more comfortably and for longer durations. Furthermore, the ergonomic and intuitive configuration of the laryngoscope handle 100 can assist medical professionals who do not perform intubation frequently by promoting proper technique, thereby increasing procedural success rates and reducing the risk of patient injury. It should be understood that the reference to a medical professional includes an anesthesiologist, emergency physician, and various other practitioners expected to serve as first responders in emergency cases requiring advanced airway management.

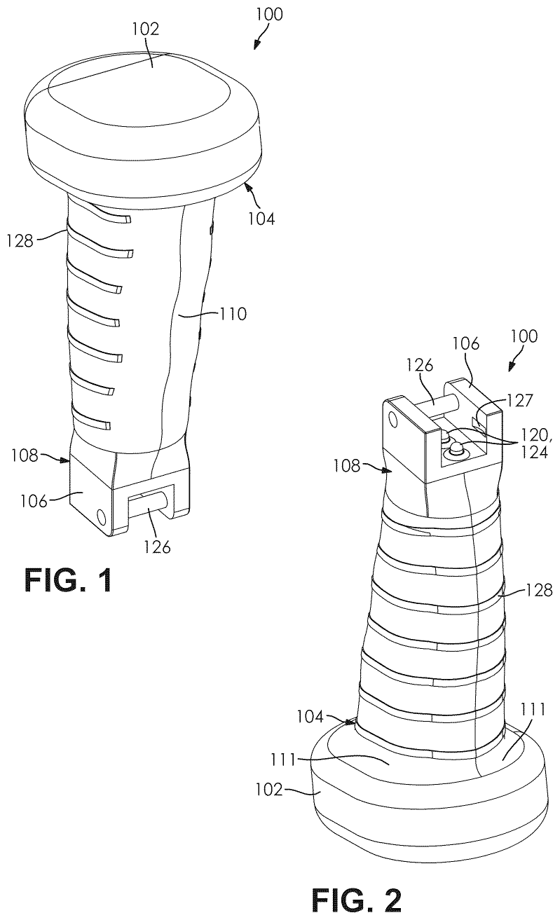

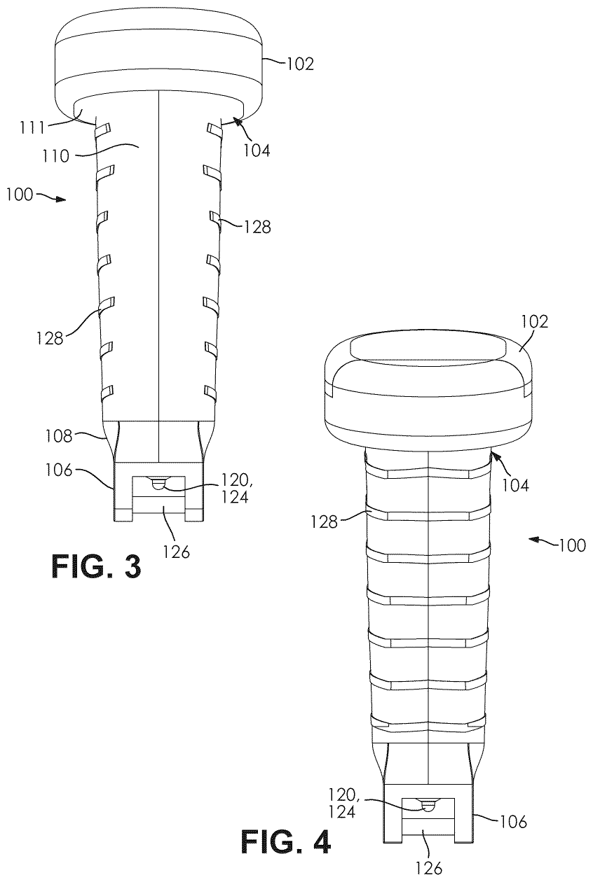

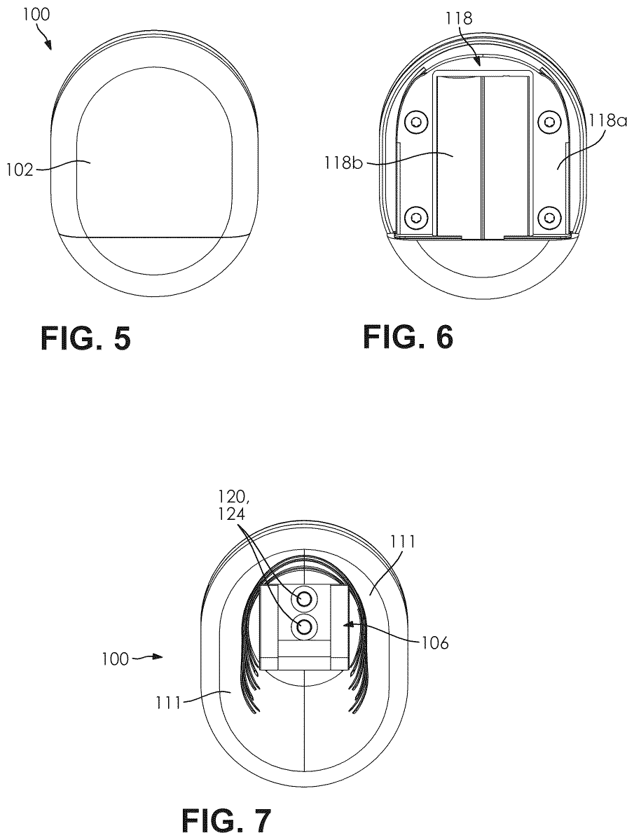

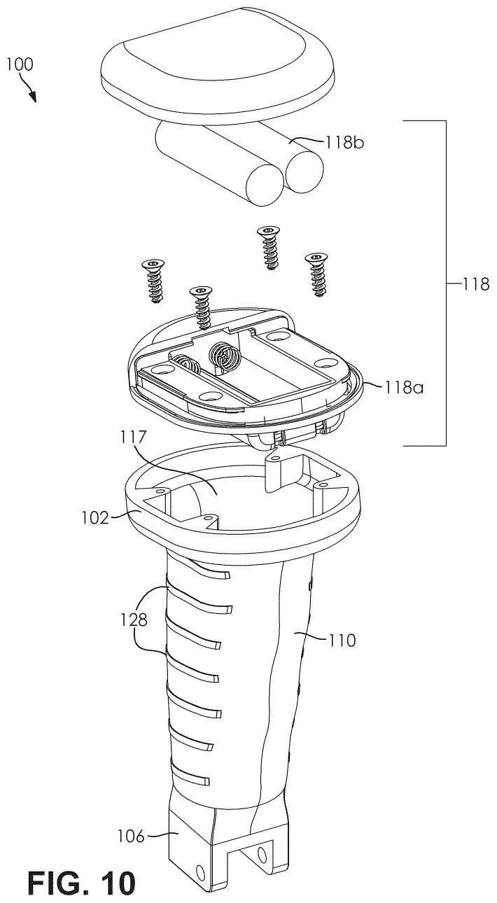

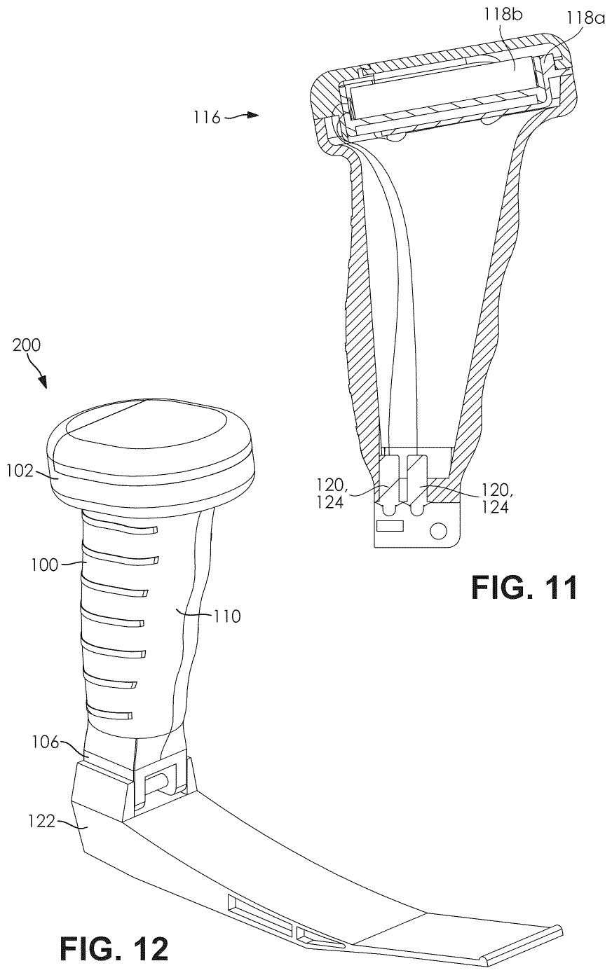

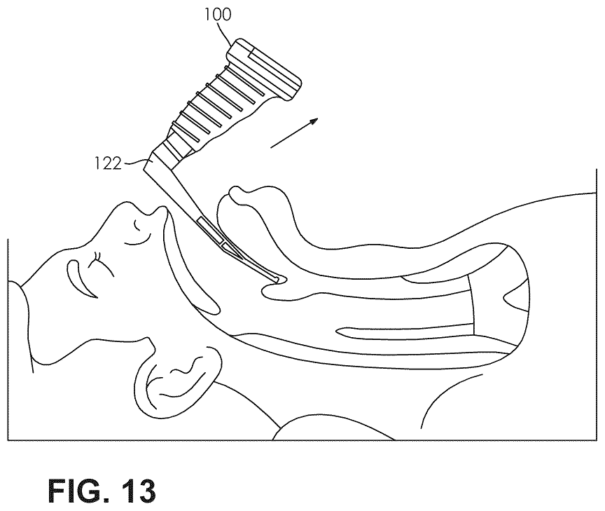

Referring now to the drawings, in certain embodiments, and with reference to FIG. 1-11, a laryngoscope handle 100 is provided. The laryngoscope handle 100 can include a cap 102 disposed at a first end 104, a blade interface 106 disposed at a second end 108 opposite the cap 102, and a gripping portion 110 positioned between the cap 102 and the blade interface 106. It should be understood that the blade interface 106, for example, can operate as a blade mounting region. The cap 102 can extend outward from the gripping portion 110 at the first end 104 and can be angled relative to a longitudinal axis A of the laryngoscope handle 100 in a manner different from the blade interface 106. This outward and angled configuration of the cap 102 can define a surface 111 against which a medical professional may abut their hand during use of the laryngoscope handle 100. By providing a point of contact, i.e., the surface 111 for the hand, the cap 102 can facilitate improved mechanical leverage and handling stability, promoting an upward lifting motion during direct or indirect laryngoscopy and endotracheal intubation. Additionally, this angled cap 102 configuration can help discourage undesirable levering or tilting of the laryngoscope handle 100 against the patient, thereby improving control.

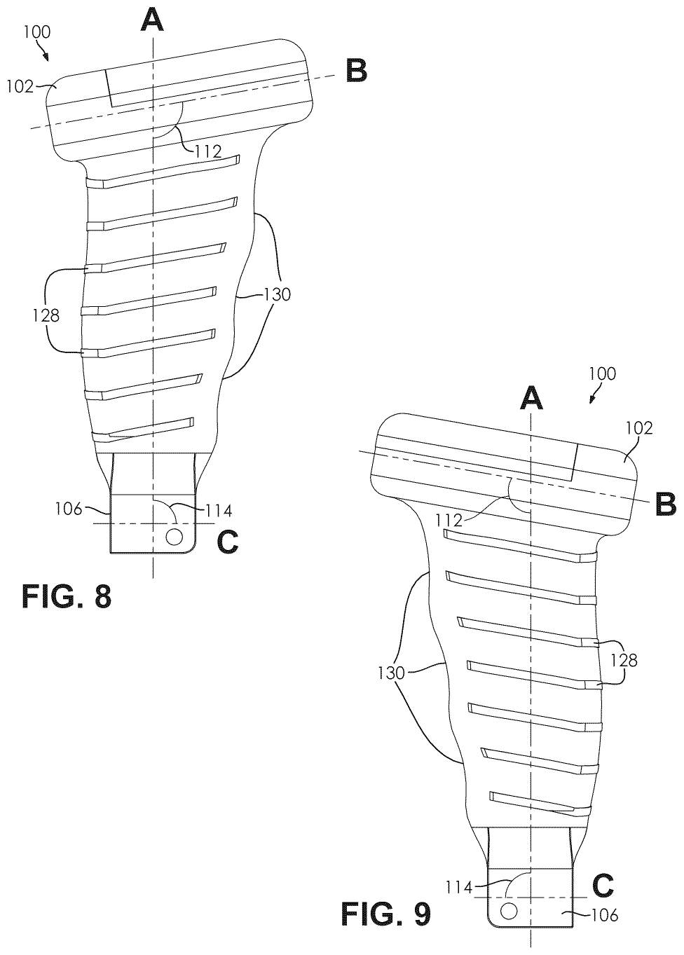

Referring now to FIGS. 8 and 9, the cap 102 can be positioned at a first angle 112 relative to the longitudinal axis A of the laryngoscope handle 100. The first angle 112 can be defined between a longitudinal axis B of the cap 102 and the longitudinal axis A of the laryngoscope handle 100. In certain embodiments, the first angle 112 can be greater than a second angle 114 formed between the longitudinal axis C of the blade interface 106 and the longitudinal axis A of the laryngoscope handle 100. By positioning the cap 102 at a greater angle relative to the longitudinal axis A of laryngoscope handle 100 than the blade interface 106, the configuration can promote an upward lifting motion. Specifically, the orientation of the cap 102 can encourage a medical professional to apply force in an upward and outward direction, rather than in a tilting or levering manner, during direct or indirect laryngoscopy and endotracheal intubation. This upward and outward lifting motion can help to maintain optimal visualization of the vocal cords and militate against injury to the patient's oral structures such as chipped or broken teeth and internal soft tissue injury.

In certain embodiments, the first angle 112 formed between the longitudinal axis B of the cap 102 and the longitudinal axis A of the laryngoscope handle 100 can be greater than 90 degrees. This configuration can result in the cap 102 extending obliquely away from the laryngoscope handle 100 in a direction that is ergonomically aligned with the natural pushing motion of a medical professional's hand during use. By having the first angle 112 exceed 90 degrees, the cap 102 can provide a rearward-facing abutment surface 111 against which the hand can be braced to enhance control and reduce slippage during the lifting phase of intubation.

The first angle 112 can be within a range from approximately 91 degrees to approximately 179 degrees. This broad range can allow for various design configurations while still achieving the functional objective of encouraging upward and outward lifting. A more obtuse angle for the cap 102 can provide a more pronounced abutment surface 111 for the medical professional's hand, thereby accommodating different hand positions, grip preferences, and procedural techniques. Additionally, medical professionals can have varying anatomical features such as hand size, wrist flexibility, or forearm strength that may make it difficult to use laryngoscopes. This angled configuration of the cap 102 can help address these ergonomic challenges by providing a structure that promotes proper lifting mechanics and reduces reliance on awkward or strain-inducing wrist angles. This can ultimately lead to more consistent intubation technique being applied by a diverse range of medical professionals when using the laryngoscope handle 100.

In certain embodiments, the first angle 112 can be in the range from approximately 100 degrees to approximately 110 degrees. First angles 112 within this range can align well with the anatomical position of the medical professional's wrist and forearm during direct or indirect laryngoscopy and endotracheal intubation. In this way, the laryngoscope handle 100 can militate against hand fatigue and can improve procedural precision.

The blade interface 106 can be oriented to form a second angle 114 with the longitudinal axis A of the laryngoscope handle 100. In certain embodiments, the second angle 114, defined between the longitudinal axis C of the blade interface 106 and the longitudinal axis A of the handle, can be approximately 90 degrees. In such configurations, the blade interface 106 can be orthogonal to the gripping portion 110 of the handle. This orthogonal arrangement can facilitate the directionality of force application during direct or indirect laryngoscopy and endotracheal intubation, complementing the upward and outward motion encouraged by the angled cap 102.

It should be understood that the cap 102 can thus be positioned at an angle that is specifically configured to promote proper lifting technique during direct or indirect laryngoscopy and endotracheal intubation. By encouraging the medical professional to lift upward and outward along a trajectory that aligns with the intended axis of movement, the cap 102 can militate against the likelihood of undesirable mechanical leverage against the patient's teeth or soft tissue structures. The configuration of the cap 102 can thereby support a more controlled and stable handling of the laryngoscope handle 100, even for a medical professional with limited experience or training in tracheal intubation.

In certain embodiments, the laryngoscope handle 100 can further comprise an illumination system 116. The illumination system 116 can be configured to provide visual illumination to a region of interest during tracheal intubation procedures. Illumination can be particularly useful for enhancing visualization of anatomical structures such as the epiglottis, vocal cords, and surrounding tissue, thereby supporting accurate and effective tracheal intubation. The inclusion of an illumination system 116 can reduce the need for external light sources and can streamline procedural workflow in a clinical setting.

In certain embodiments, the illumination system 116 can include a power supply 118 and a light source 120, as shown in FIGS. 2-4 and 6-7. The power supply 118 can be configured to energize the light source 120, which can emit visible light through or in conjunction with an attached laryngoscope blade 122. The light source 120 can include, for example, a light-emitting diode (LED), which can be selected for its efficiency, brightness, and compact size. The light source 120 may be controlled by a switch, allowing the medical professional to adjust the level of illumination as needed during direct or indirect laryngoscopy and endotracheal intubation. The power supply 118 can include a battery tray 118a and one or more batteries 118b or other energy storage components suitable for medical device applications. One having ordinary skill in the art can select a suitable power supply 118 within the scope of the present disclosure.

The illumination system 116 can be disposed within an interior 117 of the laryngoscope handle 100. In such configurations, the internal placement of the illumination system 116 can protect it from damage, minimize bulk, and maintain a streamlined external profile of the laryngoscope handle 100. Integrating the illumination system 116 within the laryngoscope handle 100 can also simplify sterilization and reduce interference with the medical professional's grip or line of sight during use.

In certain embodiments, the power supply 118 can be housed within the cap 102 of the laryngoscope handle 100. By positioning the power supply 118 in the cap 102, the design can take advantage of otherwise unused internal volume, maintain weight balance along the laryngoscope handle 100, and provide convenient access for battery replacement or recharging.

In certain embodiments, the blade interface 106 of the laryngoscope handle 100 can be configured to connect to differently configured laryngoscope blades 122. This adaptability can allow the laryngoscope handle 100 to accommodate a variety of blade geometries, sizes, and functional designs, thereby enhancing its utility. The ability to interchangeably couple with various laryngoscope blades 122 can support both direct and indirect laryngoscopy techniques, as well as pediatric and adult applications, depending on the clinical need. For example, laryngoscope blades 122 can include a Welch Allyn English MacIntosh Fiber-Optic Laryngoscope Blade, manufactured and marketed by Welch Allyn, headquartered in Skaneateles Falls, New York; a HEINE Classic+ Miller F.O. Laryngoscope Blade, manufactured and marketed by HEINE, headquartered in Gilching, Germany; and a Ri-standard Miller Laryngoscope Blade, manufactured and marketed by Rudolf Riester GmbH, headquartered in Jungingen, Germany.

The blade interface 106 can include a bar 126 that is disposed orthogonally to the longitudinal axis of the laryngoscope handle 100. The orthogonal orientation of the bar 126 can provide a transverse axis about which a laryngoscope blade 122 may be mounted or pivoted. This configuration can facilitate a secure coupling of the laryngoscope blade 122 while also supporting desired movement or positioning of the laryngoscope blade 122 during use. In certain embodiments, the bar 126 can also serve as a mechanical anchor point for various locking or retention mechanisms that help to ensure a stable coupling between the laryngoscope blade 122 and the laryngoscope handle 100.

In certain embodiments, the laryngoscope blade 122 can be configured to pivot about the bar 126 relative to the laryngoscope handle 100 between a first position and a second position (not shown). The first position can correspond to a folded or stowed orientation for storage or transport, while the second position can correspond to a deployed or operational orientation for performing tracheal intubation. A pivotable connection can provide mechanical convenience, reduce the storage footprint of the device, and improve readiness for use by allowing transition between non-use and operational states.

In certain embodiments, the blade interface 106 can include a post that is configured to receive a corresponding hook (not shown) of the laryngoscope blade in accordance with the ISO 7376 standard in effect on the filing date of this application. Conformance with ISO 7376 can ensure compatibility with a wide range of standardized laryngoscope blades commonly used in clinical practice. The post-and-hook interface can promote secure engagement, mechanical stability, and reliable alignment of the laryngoscope blade 122 relative to the laryngoscope handle 100 during use.

The blade interface 106 can include both the bar 126 and a spring-loaded locking tab (not shown) configured to secure the laryngoscope blade 122 upon rotational engagement. In such configurations, once the hook of a compatible laryngoscope blade 122 is mounted onto the bar and rotated into position, the spring-loaded tab can automatically engage with a corresponding recess or shoulder on the laryngoscope blade 122. This engagement can provide tactile and audible feedback that the laryngoscope blade 122 is securely locked in place, while also resisting unintended detachment during use.

The blade interface 106 can include various aspects in coupling the laryngoscope blade 120 thereto. On one hand, the blade interface 106 can include a snap-fit recess (not shown) configured to receive and retain a projection of the laryngoscope blade 122. The snap-fit design can allow for rapid and tool-free attachment and detachment of the laryngoscope blade 122 while maintaining a secure fit during operation. The snap-fit recess can be dimensioned to receive standard or proprietary projections and can include resilient features to accommodate minor dimensional variations among blades. One the other hand, the blade interface 106 can include a twist-lock connection configured to rotationally secure the laryngoscope blade 122. The twist-lock mechanism can allow a user to insert the laryngoscope blade 122 into the interface and rotate it into a locked position, thereby simplifying the attachment process and providing a secure coupling.

In certain embodiments, the blade interface 106 of the laryngoscope handle 100 can include an optical interface 124 configured to direct light from a light source disposed within the laryngoscope handle 100 into a fiber-optic bundle of the laryngoscope blade 122. The optical interface 124 can include one or more optical elements, such as lenses, light guides, or transparent windows aligned to channel and efficiently couple light from the illumination system 116 to the proximal end of the fiber-optic bundle of the laryngoscope blade 122. The optical interface 124 can include alignment features to ensure proper optical coupling between the laryngoscope handle 100 and the laryngoscope blade 122, reducing light loss and maximizing visibility for the medical professional.

The blade interface 106 can be configured to receive a laryngoscope blade 122 that is compliant with the ISO 7376 fiber-optic laryngoscope standard in effect on the filing date of this application. Compliance with this international standard can ensure mechanical and optical compatibility with a wide range of fiber-optic laryngoscope blades used in clinical practice. The standard may define dimensions, connection geometries, and optical coupling requirements, among other characteristics.

In certain embodiments, the blade interface 106 can include a recess 127 configured to provide audible or tactile feedback to confirm full engagement of the laryngoscope blade 122 with the laryngoscope handle 100. The recess 127 can accept a spring-loaded element, snap feature, or other mechanical structure that temporarily resists motion until a threshold is overcome resulting in a clicking sound or palpable resistance that indicates proper seating of the laryngoscope blade 122. This audible or tactile feedback can militate against the risk of incomplete attachment, which could otherwise compromise procedural safety or effectiveness. It should be understood that the recess 127 can be designed to work in conjunction with other securing features such as locking tabs or twist-lock connections to provide redundant assurance of engagement.

With reference to FIGS. 8-9, the gripping portion 110 of the laryngoscope handle 100 can include features configured to enhance ergonomic comfort and improve the stability of the grip by the medical professional during use. For example, the gripping portion 110 can include ribbing 128 that extends along all or part of the handle surface. The ribbing 128 can be formed as raised linear projections or contours that run longitudinally, circumferentially, or in a helical pattern around the gripping portion. In certain embodiments, the gripping portion 110 can include ridges, defined as elevated lines or bands that protrude from the handle surface. The ridges can be provided in uniform or variable spacing, and can function similarly to ribbing by interrupting smooth surfaces and creating multiple contact points for the hand. These features can reinforce a non-slip interface between the user's hand and the handle, contributing to safer and more confident manipulation of the device.

In certain embodiments, the gripping portion 110 can further include a plurality of spaced finger grooves 130, as shown in FIGS. 8-9. The finger grooves 130 can be contoured recesses dimensioned to receive the fingers of a medical professional during use. The finger grooves 130 can be spaced apart at intervals that correspond to average finger spacing, and may extend transversely or obliquely relative to the longitudinal axis of the laryngoscope handle 100. These finger grooves 130 can improve ergonomic comfort, distribute gripping forces more evenly, and facilitate intuitive hand placement, which may be particularly beneficial for a medical professional who infrequently performs laryngoscopy.

The gripping portion 110 can include surface texturing that provides tactile feedback and frictional resistance. The texturing can take the form of fine granular patterns, micro-patterned projections, or molded surface irregularities that enhance grip security. Texturing can be especially advantageous in high-stress, time-sensitive scenarios, allowing the user to maintain control of the handle even when gloves are worn or when the handle becomes slick due to fluid exposure.

In certain embodiments, the gripping portion 110 can include stippling, which refers to a surface finish comprising a dense pattern of small, raised dots or bumps. Stippling can be molded, machined, or applied as a coating to the outer surface of the handle. This stippled texture can produce a high-friction surface that resists slippage without significantly altering the overall geometry of the laryngoscope handle 100. The stippling can also provide tactile differentiation, enabling the medical professional to quickly locate and orient the laryngoscope handle 100 by feel alone.

In certain embodiments, the gripping portion 110 of the laryngoscope handle 100 can be formed from a medical-grade material, such as silicone. The use of medical-grade silicone can provide a number of advantages, including a soft-touch surface, chemical resistance, and biocompatibility. Silicone can also offer resilience and flexibility, which may enhance the comfort and control experienced by a medical professional during use, particularly when procedures are lengthy or require precise manipulation.

The gripping portion 110 can include contouring configured to provide ergonomic handling. The contouring can be formed to follow the natural shape of hand of a medical professional, thereby supporting a neutral wrist posture and reducing the effort required to maintain a secure grip. Such ergonomic features can reduce hand fatigue and promote more stable and controlled operation, even for medical professionals who may not perform laryngoscopy on a routine basis.

In certain embodiments, the laryngoscope handle 100 can be formed as a unitary body. Forming the laryngoscope handle 100 as a single, continuous structure can minimize assembly complexity and eliminate seams or junctions, thereby simplifying a cleaning process. A unitary construction can be achieved through molding, casting, or additive manufacturing techniques, depending on the selected material. The laryngoscope handle 100 can also be formed as a multi-component assembly. A modular approach can facilitate manufacturing, maintenance, and component replacement. The multi-component assembly can include a first component, a second component, and a plurality of alignment pins. Each alignment pin can extend from the first component and be configured to engage a corresponding hole in the second component. The alignment pins can maintain axial alignment between the components during assembly and ensure structural integrity and functional consistency once the handle is assembled.

The laryngoscope handle 100 can be formed from a medical-grade material. Medical-grade materials can be specifically selected to meet stringent requirements for biocompatibility, sterilizability, and mechanical strength in clinical settings. These materials can be compatible with high-level disinfection protocols and resistant to degradation from bodily fluids, cleaning agents, or repeated use. Examples of suitable medical-grade materials include, stainless steel, aluminum, titanium, polycarbonate, polyethersulfone, polysulfone, polyetherimide, silicone, polypropylene, and acrylonitrile butadiene styrene (ABS). Each of these materials can offer distinct advantages depending on the desired characteristics of the handle. For instance, stainless steel may be preferred for its durability and ease of sterilization, while polycarbonate and silicone may be favored for their lightweight nature and improved tactile properties. A person having ordinary skill in the art can select a suitable medical-grade material for the laryngoscope handle 100 within the scope of the present disclosure.

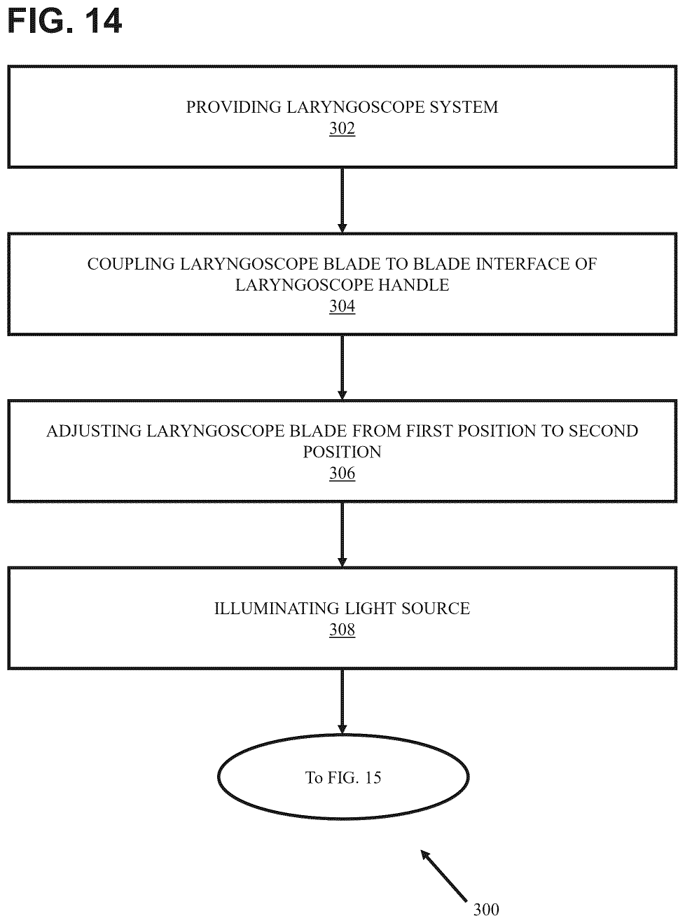

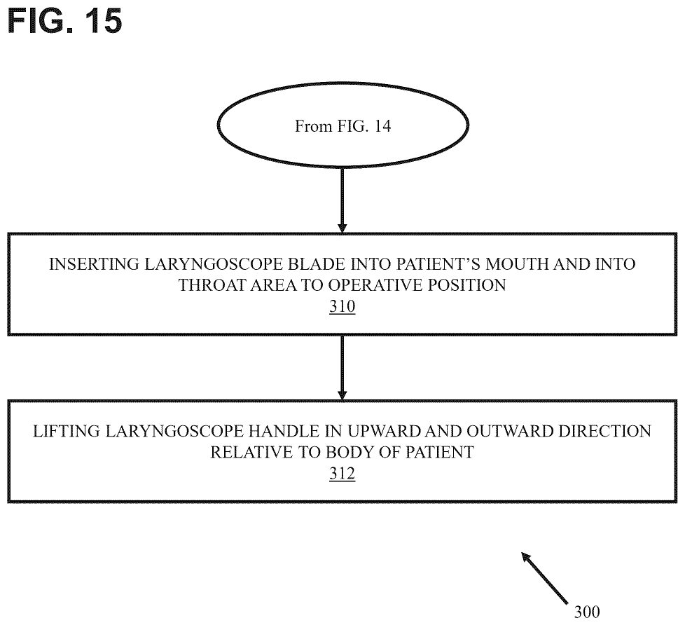

In certain embodiments, and with reference to FIGS. 14 and 15, a method 300 of using a laryngoscope system 200 for tracheal intubation is provided. The method 300 can include a step 302 of providing the laryngoscope system 200, which can include a laryngoscope handle 100 and a laryngoscope blade 122, as described herein. The method 300 can further include a step 304 of coupling the laryngoscope blade 122 to the blade interface 106 of the laryngoscope handle 100. The blade interface 106 can be configured to receive and secure various types of laryngoscope blades 122 via mechanical and electrical engagement.

The method 300 can include a step 306 of adjusting the laryngoscope blade 122 from a first position to a second position. In certain embodiments, this adjustment can involve pivoting the laryngoscope blade 122 about a bar or similar interface feature to align the laryngoscope blade 122 in preparation for insertion. The method 300 can also include a step 308 of illuminating a light source associated with the laryngoscope system 200.

The method 300 can include a step 310 of inserting the laryngoscope blade 122 into the mouth of the patient and advancing it into the throat area to reach an operative position. Once the laryngoscope blade 122 is positioned, the method 300 can include a step 312 of lifting the laryngoscope handle 100 in an upward and outward direction relative to the patient's body. This lifting motion can be facilitated by the structural configuration of the handle, particularly the cap 102, which can be angled differently than the blade interface 106 relative to a longitudinal axis of the laryngoscope handle 100. This angular differentiation can be configured to encourage a lifting path that promotes visualization of the vocal cords while discouraging a levering or tilting motion that could result in contact with the patient's teeth or soft tissues.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms, and that neither should be construed to limit the scope of the disclosure. In some example embodiments, well-known processes, well-known device structures, and well-known technologies are not described in detail. Equivalent changes, modifications and variations of some embodiments, materials, compositions and methods can be made within the scope of the present technology, with substantially similar results.

What is claimed is:

1. A laryngoscope handle for use with a laryngoscope blade, comprising:
a cap disposed at a first end;
a blade interface disposed at a second end, opposite the cap; and
a gripping portion disposed between the cap and the blade interface,
wherein:
the cap extends outward from the gripping portion at the first end, and
the cap is angled differently than the blade interface relative to a longitudinal axis of the laryngoscope handle.

2. The laryngoscope handle of claim 1, wherein the cap is positioned at a first angle relative to the longitudinal axis of the laryngoscope handle that is greater than a second angle formed between a longitudinal axis of the blade interface and the longitudinal axis of the laryngoscope handle thereby encouraging a medical professional to lift upward on the laryngoscope handle during tracheal intubation.

3. The laryngoscope handle of claim 2, wherein a longitudinal axis of the cap and the longitudinal axis of the laryngoscope handle form the first angle, the first angle greater than 90 degrees.

4. The laryngoscope handle of claim 1, wherein a longitudinal axis of the blade interface and the longitudinal axis of the laryngoscope handle are orthogonal to each other.

5. The laryngoscope handle of claim 1, wherein the cap defines a surface where contact with the surface and the cap cooperate to encourage a medical professional to lift upward on the laryngoscope handle during tracheal intubation.

6. The laryngoscope handle of claim 1, further comprising an illumination system.

7. The laryngoscope handle of claim 6, wherein the illumination system includes a power supply and a light source.

8. The laryngoscope handle of claim 6, wherein the illumination system is disposed within an interior of the laryngoscope handle.

9. The laryngoscope handle of claim 1, wherein the blade interface includes an optical interface configured to direct light from a light source into a fiber-optic bundle of a laryngoscope blade.

10. The laryngoscope handle of claim 9, wherein the optical interface includes lenses, light guides, or transparent windows aligned to channel light a proximal end of the fiber-optic bundle of the laryngoscope blade.

11. The laryngoscope handle of claim 1, wherein the blade interface includes a bar disposed orthogonally to the longitudinal axis of the laryngoscope handle.

12. The laryngoscope handle of claim 11, wherein the bar of the blade interface is configured to allow a laryngoscope blade coupled thereto to pivot about the bar between a first position and a second position.

13. The laryngoscope handle of claim 7, wherein the blade interface includes an optical interface configured to direct light from the light source into a fiber-optic bundle of the laryngoscope blade.

14. The laryngoscope handle of claim 1, wherein the blade interface includes a recess configured to audibly or tactilely confirm full engagement of the laryngoscope blade.

15. The laryngoscope handle of claim 1, wherein the gripping portion includes a plurality of spaced finger grooves.

16. The laryngoscope handle of claim 1, wherein the gripping portion includes texturing.

17. A laryngoscope comprising:
the laryngoscope handle according to claim 1; and
a laryngoscope blade configured to couple to the blade interface of the laryngoscope handle.

18. A laryngoscope system for use during tracheal intubation, comprising:
a laryngoscope handle including:
a cap disposed at a first end,
a blade interface disposed at a second end, opposite the cap,
a gripping portion disposed between the cap and the blade interface,
an illumination system including:
a power supply, and
a light source,
wherein:
the cap extends outward from the gripping portion at the first end, and
the cap is angled differently than the blade interface relative to a longitudinal axis of the laryngoscope handle thereby encouraging a medical professional to lift upward on the laryngoscope handle during tracheal intubation;
the gripping portion includes a plurality of spaced finger grooves; and
a laryngoscope blade.

19. The laryngoscope system of claim 18, wherein the laryngoscope blade is comprised by a plurality of laryngoscope blades.

20. A method of using a laryngoscope system for tracheal intubation, the method comprising:
providing the laryngoscope system of claim 18;
inserting the laryngoscope blade into a mouth of a patient and into a throat area to an operative position;
lifting the laryngoscope handle in an upward and outward direction relative to a body of the patient, wherein lifting is facilitated by the cap being angled differently than the blade interface relative to the longitudinal axis of the laryngoscope handle thereby promoting exposure of vocal cords and discouraging tilting or levering of the laryngoscope blade against a patient's teeth or soft tissues.
